(19) United States
(12) Patent Application Publication
(10) Pub. No.: US 2003/0077573 A1
(43) Pub. Date: Apr. 24, 2003

(54) Methods of identifying compounds useful in the inhibition of neurodegenerative disease

(76) Inventors: Daniel Paris, Tampa, FL (US); Terrence C. Town, Tampa, FL (US); Michael Mullan, Tampa, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/305,508

(22) Filed: Nov. 27, 2002

Related U.S. Application Data
(63) Continuation of application No. 09/603,892, filed on Jun. 26, 2000.
(60) Provisional application No. 60/140,797, filed on Jun. 25, 1999.

(51) Int. Cl. C12Q 1/00

(52) U.S. Cl. 435/4

(57) ABSTRACT

Methods for screening compounds for use in the treatment of neurodegenerative diseases such as Alzheimer's disease, multiple sclerosis, prion diseases, Huntington's disease, and Creutzfeldt-Jakob disease, where the screening methods include (a) measuring the vasoactivity of blood vessels when contacted sequentially with a β-amyloid peptide, phenylephrine, and a compound to be screened, and (b) measuring the inflammatory response, including cytokine release, when microglial cells are contacted with a β-amyloid peptide and then with a compound to be screened. The disclosed methods rely upon a common underlying mechanism for β-amyloid peptide-induced vasoactivity, β-amyloid peptide-induced inflammatory responses of microglial cells, and the pathogenesis of neurodegenerative diseases in general and Alzheimer's disease in particular, in which cGMP levels are increased by agents that counteract these three processes.

RELATED APPLICATIONS

This application is a continuation application of U.S. Ser. No. 09/603,892; filed Jun. 26, 2000 which claims the benefit of U.S. Ser. No. 60/140,797; filed Jun. 25, 1999, which is hereby incorporated in its entirety by reference.

### FIELD OF THE INVENTION

The present invention relates generally to methods for treating Alzheimer's Disease (AD) and methods of identifying compounds useful in the inhibition of Alzheimer's Disease. More specifically, the present invention relates to methods of treating AD by reducing β-amyloid microglial-mediated inflamrnmation and β-amyloid vasoactivity via inhibition of cyclic GMP phosphodiesterases (cGMP-PDEs) or promotion of increased cGMP levels.

### BACKGROUND AND SUMMARY OF THE INVENTION

β-amyloid (Aβ) precursor proteins present in individuals with AD are generally large transmembrane proteins that are cleaved to form Aβ peptides which are deposited in brain parenchyma as senile plaques, as well as in cortical and meningeal vessels. Vascular damage and reactive gliosis are found co-localized with amyloid deposits in AD brains, suggesting that the vasculature may be a clinically significant site of AD pathology.

Experiments using intact rat aortae in a tissue bath system show that freshly solubilized Aβ₁₋₄₀ is vasoactive in vitro (similar demonstrations may be made using other blood vessels). Further, in vivo experiments demonstrate that Aβ₁₋₄₀ is vasoactive, leading to a specific decrease in cerebral blood flow. See, for example, Suo, Z. et al., Soluble Alzheimer's β-amyloid constricts the cerebral vasculature in vivo. Neurosci. Lett. 257:77-80, (1998). Therefore, increasing cerebral concentrations of Aβ could contribute to AD pathology by microvascular vasoconstriction, reducing cerebral blood flow resulting in hypoperfusion and ischaemia. See, for example, Zhang et al., Positron emission tomography in Alzheimer's disease. Neurology 36: 879-887 (1986).

Additionally, human recombinant apolipoprotein E isoforms are vasoconstrictive in vitro (E4>E3>E2) correlating with the allele-specific genetic risk conferred by APOE for both hypertension and AD (Paris D. et al., Isoform-specific vasoconstriction induced by apolipoprotein E and modulation of this effect by Alzheimer's β-amyloid peptide. Neurosci. Lett. 256: 73-76 (1998)), suggesting that AD and hypertension may have common etiological roots. Indeed, incidence of hypertension prior to diagnosis of AD is a predictive factor for AD, an example is provided by Lis, C. G., et al., Vascular dementia, hypertension, and the brain. Neurol. Res. 19:471-480 (1997), and Skoog, I., The relationship between blood pressure and dementia: a review. Biomed. Pharmacother. 51:367-375 (1997).

Previously, it has been shown that Endothelin-1 (ET-1), one of the most potent cerebrovascular vasoconstrictors, together with Nitric Oxide (NO), control cerebral vasoregulation. See, for example, Douglas, S. A., et al., Signal transduction mechanisms mediating the vascular actions of endothelin. J. Vas. Res. 34:152-164, (1997), Levin, E. R. Endothelins as cardiovascular peptides, Am. J. Neph. 16:246-251, (1996), and Ehrenreich, H., et al., New developments in the understanding of cerebral vasoregulation and vasospasm: the endothelin-nitric oxide network. Cleve. Clin. J. Med. 62:105-116, (1995). For example, ET-1 significantly reduces cGMP levels in vessels stimulated by the NO donor sodium nitroprusside (SNP). Pussard, G., et al., Endothelin-1 modulates cyclic GMP production and relaxation in human pulmonary vessels. J. Pharmacol. Exp. Ther. 274:969-75, (1995). As we have previously shown that Aβ peptides are vasoconstrictive, it is therefore desirable to determine whether Aβ modulates the NO/cGMP pathway.

Soluble guanylyl cyclase (sGC) is responsible for the synthesis of cGMP. NO stimulates sGC in underlying vascular smooth muscle cells, thereby elevating intracellular levels of cGMP and inducing relaxation of the vascular smooth muscle. Moncada, S., et al., Nitric oxide: physiology, pathophysiology, and pharmacology. Pharmacol. Rev. 43:109-142, (1991). Thus, the disclosure provided herein shows the role of sGC in the vasoactivity mediated by Aβ. In order to determine the involvement of sGC in the vasoactivity of Aβ, a highly selective inhibitor of sGC, 1H-[1,2,4]oxadiazolo[4,3,-α]quinoxalin-1-one (ODQ, 49) is used. The vasoconstriction induced by ET-1 is synergistically enhanced after Aβ or ODQ treatment, but is only additive (as opposed to statistically interactive) with Aβ and ODQ co-treatment, demonstrating that Aβ is not able to modulate the activity of sGC. YC-1, an NO-independent activator of sGC, is only able to reduce Aβ induced vasoconstriction in an additive manner, further confirming that Aβ vasoactivity is not mediated via sGC. In addition, inhibition of nitric oxide synthase (NOS) activity by L-NAME enhances ET-1 induced vasoconstriction, yet this effect is also merely additive in conjunction with Aβ, showing that Aβ vasoactivity does not result from an alteration of NOS activity or NO production.

Having shown that an alteration of the NO-mediated synthesis of cGMP is not involved in Aβ vasoactivity, the implication of the breakdown of cGMP, or its hydrolysis, is then examined. Cyclic GMP is primarily degraded through phosphodiesterases (cGMP-PDEs). Dipyridamole, a specific inhibitor of type V cGMP-PDE, is used to test the possible involvement of cGMP-PDE in Aβ vasoactivity. No significant difference in dipyridamole-induced relaxation between Aβ-treated and control vessels is found, showing that dipyridamole blocks the opposition to relaxation normally induced by Aβ. It is also shown that dipyridamole is able to block Aβ enhancement of ET-1-induced constriction in a statistically interactive manner, suggesting that, taken together with the finding that Aβ does not modulate sGC or NOS activity, Aβ stimulates cGMP degradation. To further confirm this hypothesis, cGMP levels in Aβ-treated aortae are examined. It is found that cGMP levels are reduced in Aβ-treated vessels compared to untreated controls but cAMP levels remain unchanged, suggesting that Aβ specifically induces cGMP degradation. These data suggest that Aβ's vasoactivity is mediated through a specific signal transduction pathway involving cGMP. Furthermore, the suggestion arises that this transduction pathway may generally mediate Aβ's bioactivity in different cell types.

To further evaluate this hypothesis, Aβ's effect on cultured microglia is examined. It is found that Aβ induces a pro-inflamrnmatory response in microglial as evidenced by increased release of leukotriene B4 (LTB4). It is shown that dipyridamole and selective cGMP-elevating agents are able to block this Aβ-induced effect. Taken together, these data show that Aβ's effects on both isolated vessels and cultured microglia can be inhibited via a cGMP-dependent mechanism, suggesting that Aβ's bioactivity is mediated via a common signal transduction pathway in different systems.

The investigation of microglial activation is particularly germane as a pathological glial cell activation, which involves transformation of microglia from a ramified to a reactive state exhibiting neurotoxic properties, is a possible contributor to the pathogenesis of various neurodegeneratives diseases such as Alzheimer's disease, multiple sclerosis, Huntington's disease, cerebral amyloid angiopathy, ischemialhyperfusion, prion diseases, and Creutzfeldt-Jakob disease. Reactive microglia release pro-inflammatory cytokines such as tumor necrosis factor alpha (TNF-α) and interleukin-1β, which have been implicated in neural cell injury, suggesting a mechanism whereby activated microglia may contribute to neurodegeneration. Inflammation is also implicated in other conditions than neurodegeneration: for example, other inflammatory diseases include rheumatoid arthritis, atheroscerosis, hypertension, hypercholesterolemia, arteriosclerosis, meningitis, and septicemia, and the methods of the present invention are applicable to these diseases also. Reactive microglia also release nitric oxide, glutamate, and eicosanoid products. Bacterial lipopolysaccharide (LPS) is a potent activator of professional macrophages such as microglia, where it induces a generalized inflammatory response, providing for an in vitro model of microglial activation. LPS treatment of microglia is accompanied by stimulation of nitric oxide (NO) production, which is commonly used as an indicator of microglial activation. However, the role of NO in microglial activation is unclear, as NO can exert both pro- and anti-inflammatory effects. NO is a known stimulator of soluble guyanylyl cyclase, which results in increased intracellular cGMP concentration in a variety of cell types, including neuronal cells. Luo, D., et al., Nitric oxide-dependent efflux of cGMP in rat cerebellar cortex: an in vivo microdialysis study, J. Neurosci., 14, 263-71 (1994). It is therefore desirable to examine the role of NO and cGMP-elevating agents on regulating microglial activation.

Dipyridamole has been used as a coronary vasodilator and, as a recent finding suggests, can be used as an antianginal agent. Picano, E. et al., Chronic oral dipyridamole as a ‘novel’ antianginal drug: the collateral hypothesis. Cardiovas. Res. 33:666-670, (1997). Moreover, patients with refractory pulmonary hypertension who fail to respond to inhaled NO demonstrate a response to combined therapy of inhaled NO plus dipyridamole. Fullerton, D. A. et al., Effective control of refractory pulmonary hypertension after cardiac operations. J. Thor. Cardiovas. Surg. 113:363-368, (1997). It is desirable to determine whether dipyridamole could be beneficial in the treatment of AD by opposing this effect.

It is disclosed herein that Aβ displays pro-inflammatory properties on microglia by inducing LTB4 release. Thus, dipyridamole, by blocking Aβ's pro-inflammatory effect, can also be beneficial in reducing the reactive gliosis associated with AD. Increasing evidence suggests that oxidative damage to proteins and other macromolecules is also a salient feature of the pathophysiology of Alzheimer's disease. See, for example, Famulari, A. L. et al., The antioxidant enzymatic blood profile in Alzheimer's and vascular diseases. Their association and a possible assay to differentiate demented subjects and controls. J. Neurol. Sci. 141:69-78, (1996); Markesbery, W. R. Oxidative stress hypothesis in Alzheimer's disease. Free Rad. Bio. Med. 23:134-147, (1997); and Thome, J., W. et al. Oxidative-stress associated parameters (lactoferrin, superoxide dismutases) in serum of patients with Alzheimer's disease. Life Sci. 60:13-19, (1997). It has also been demonstrated that dipyridamole displays anti-oxidant properties. See, for example Iuliano, L., et al., A potent chainbreaking antioxidant activity of the cardiovascular drug dipyridamole. Free Rad. Biol. Med. 18:239-247, (1995); and luliano, L., et al., Protection of low density lipoprotein oxidation at chemical and cellular level by the antioxidant drug dipyridamole. Brit. J. Pharmacol. 119:1438-1446, (1996).

Propentofylline, a non-specific cGMP/cAMP-PDE inhibitor, has been used as a novel therapy in AD. See, for example Kittner, B., et al. Clinical trials in dementia with propentofyllhne. Ann. N.Y. Acad. Sci. 826:307-316, (1997); Marcusson, J., et al., A 12-month, randomized, placebo-controlled trial of propentofylline (HWA 285) in patients with dementia according to DSM III-R. The European Propentofylline Study Group. Dement. Geriatr. Cogn. Disord. 8:320-328, (1997); and Mielke, R. et al., Propentofylline enhances cerebral metabolic response to auditory memory stimulation in Alzheimer's disease. J. Neurol. Sci. 154, 76-82, (1998). Thus, propentofylline exhibits a protective role in slowing the progression of AD, most likely by reducing microglial activation. Based upon the data disclosed herein, the dipyridamole compound, as well as any other cGMP elevating agents, in regard to their specific opposition of microglial activation and Aβ vasoactivity and microglial inflammation, are even more viable therapeutic agents in the treatment of AD.

Therefore, according to the present invention, there is provided an assay for determining the therapeutic effectiveness of an agent on AD by treating aorta with β-amyloid peptides, adding PE, adding a therapeutic agent, and measuring mean relaxation of the aorta. In addition, an assay is provided for determining the effect of an agent on AD by treating microglial cells with β-amyloid peptides, adding a therapeutic agent, and measuring microglial activation including measuring TNF-αproduction and LBT4 production. Further, a method of screening compounds for the treatment of Alzheimer's disease by treating aortae with a potentially therapeutic agent, adding ET-1 is provided for measuring the effect of an agent on AD by treating aorta with a therapeutic agent, adding ET-1, and measuring vessel response in terms of percent vasoconstriction and cGMP levels.

Finally, the proposed therapeutic agents such as dipyridamole, other cGMP-PDE inhibitors or cGMP-elevating compounds, inhibit cGMP-PDE and/or elevate cGMP levels resulting in reduced microglial-mediated activation and inflammation thereby providing a mechanism for the treatment of AD.

### BRIEF DESCRIPTION OF THE DRAWINGS

FIG. 1 illustrates the effect of Aβ on the relaxation induced by SNP.

FIG. 2 illustrates the effect of ODQ on the relaxation induced by SNP.

FIG. 3 illustrates the effect of ODQ on Aβ-enhancement of ET-1-induced vasoconstriction.

FIG. 4 illustrates the effect of YC-1 on Aβ-enhancement of ET-1-induced vasoconstriction.

FIG. 5 illustrates the effect of L-NAME on Aβ-enhancement of ET-1-induced vasoconstriction.

FIG. 6 illustrates the effect of Aβ on the relaxation induced by dipyridamole.

FIG. 7 illustrates the effect of dipyridamole on Aβ-enhancement of ET-1 induced vasoconstriction.

FIGS. 8A and B illustrate cAMP and cGMP levels in isolated rat aortae after ET-1 or Aβ+ET-1 treatment.

FIG. 9 illustrates the effects of various treatment conditions on Aβ-induced microglial LTB4 release.

FIG. 10 illustrates the effects of various treatments on TNF-α release.

### DETAILED DESCRIPTION OF THE DRAWINGS

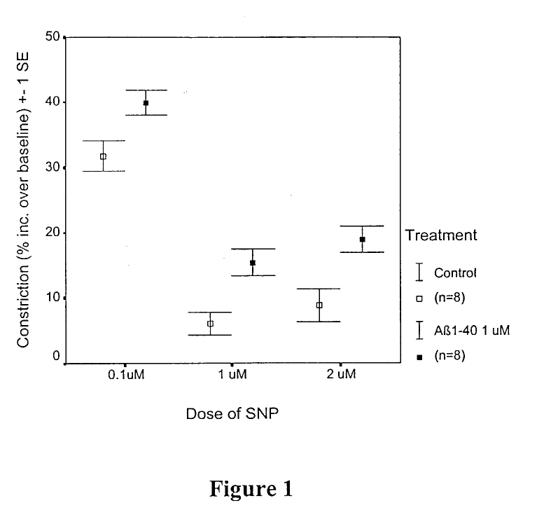

FIG. 1 is a graph showing the effect of Aβ on the relaxation induced by SNP. Control vessels and vessels pre-treated with 1 μM of freshly solubilized Aβ₁₋₄₀ are constricted with 3.5 nM of PE. Further relaxation is induced with the doses of sodium nitroprusside (SNP). Data is standardized such that the maximum value for PE-induced tension is 100% for both Aβ and control channels. Aβ (p<0.001) and SNP (p<0.001) are significant factors in the ANOVA although there is no significant interaction between them (p=0.890). Aβ decreases the induced relaxation at 0.1 μM SNP (p<0.02), at 1 μM SNP (p<0.01) and at 2 μM SNP (p<0.01). N=8 for all conditions.

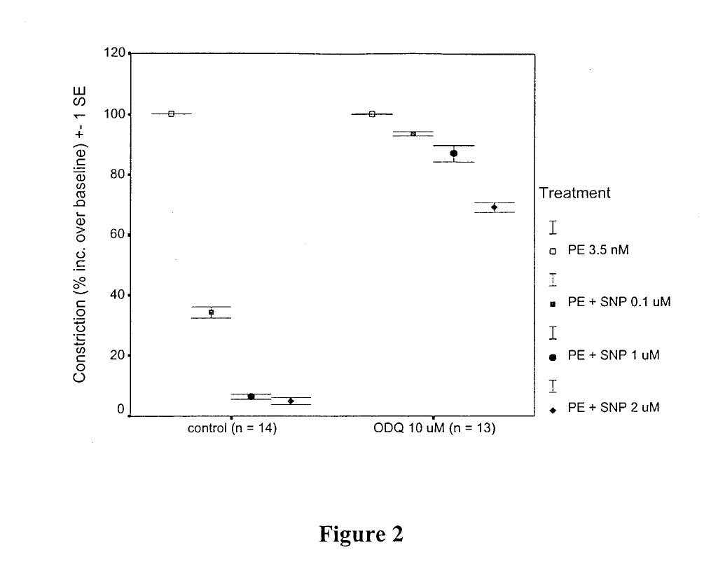

FIG. 2 is a graph showing the effect of ODQ on the relaxation induced by SNP. Vessels are pre-constricted with 3.5 nM of PE. A preferred range for PE concentration in the methods of this invention is 0.5 nM to 10 nM. Most preferably the PE concentration is 3.5 nM. Following stabilization of vasotension, thirteen vessels are treated with 10 μM of ODQ. Fourteen control vessels are untreated. All vessels are then relaxed with three different doses of SNP (0.1 μM, 1 μM, and 2 μM). Data is standardized such that the maximum PE or PE+ODQ-induced constriction is fixed to 100%. Both ODQ (p<0.001) and SNP (p<0.001 ) are significant main effects in the ANOVA and there is a significant interactive term between these treatments (p<0.001). One-way ANOVA reveals significant between-groups differences (p<0.001) among treatment conditions, and post-hoc comparison between ODQ-treated and untreated vessels for each dose of SNP showed significant differences (p<0.001).

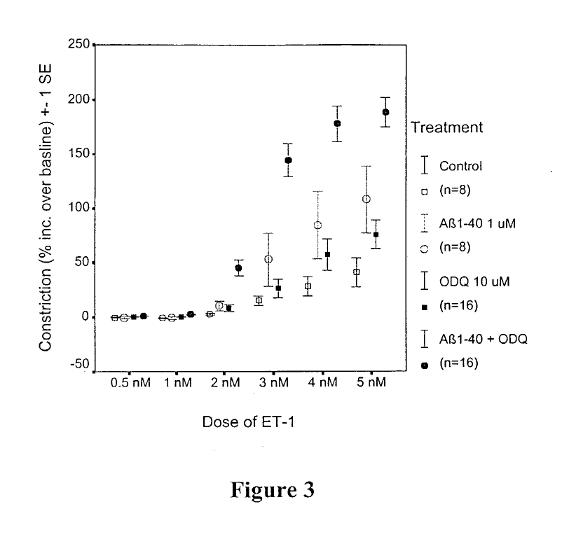

FIG. 3 is a graph demonstrating the effect of ODQ on Aβ-enhancement of ET-1 induced vasoconstriction. Aortic rings are treated with 1 μM of freshly solubilized Aβ₁₋₄₀ (N=8), 10 μM of ODQ (N=16), or ODQ+Aβ (N=16) for five minutes prior to the addition of a dose range of ET-1. Results are expressed as the mean±1 SE of the percentage vasoconstriction increase over baseline. Significant main effects with Aβ (p<0.001), ODQ (p<0.001), and ET-1 (p<0.001) and an interaction between ET-1 and either Aβ (p<0.001) or ODQ (p<0.00 1). Treatment of the aortic rings with Aβ+ODQ gives an additive constriction which is not interactive by ANOVA (p=0.129).

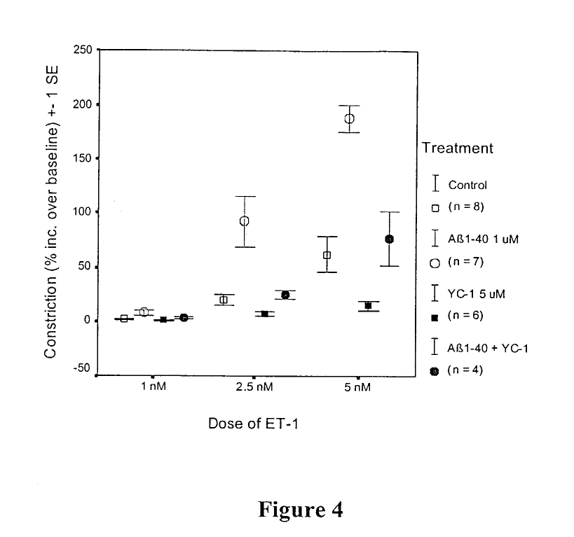

FIG. 4 is a graph showing the effect of YC-1 on Aβ-enhancement of ET-1-induced vasoconstriction. Aortic rings are treated with 1 μM of freshly solubilized Aβ₁₋₄₀ (N=7), 5 μM of YC-1 (N=6), or YC-1+Aβ (N=4) for five minutes prior to the addition of a dose range of ET-1. Results are expressed as the mean±1 SE of the percentage vasoconstriction increase over baseline. ANOVA showed significant main effects with Aβ (p<0.001), YC-1 (p<0.001), and ET-1 (p<0.001) and an interaction between ET-1 and either Aβ (p<0.001) or YC-1 (p<0.001). Treatment of the aortic rings with Aβ+YC-1 reduces vasoconstriction but is not interactive by ANOVA (p=0.179).

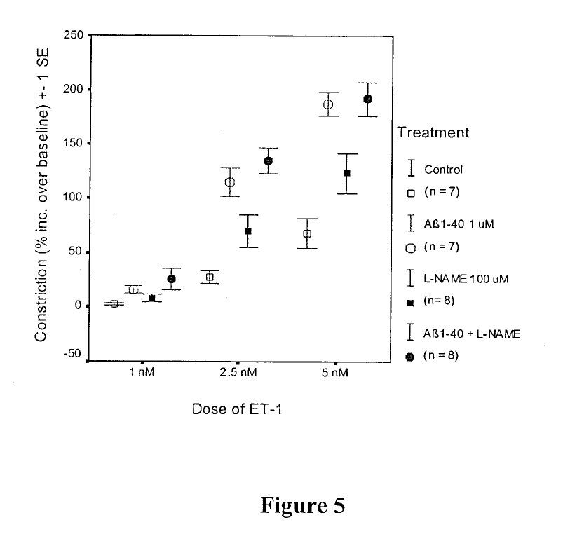

FIG. 5 is a graph showing the effect of L-NAME on Aβ-enhancement of ET-1-induced vasoconstriction. Aortic rings are treated with 1 μM of freshly solubilized Aβ₁₋₄₀ (N=7), 100 μM of L-NAME (N=8), or L-NAME+Aβ (N=8) for five minutes prior to the addition of a dose range of ET-1. Results are expressed as the mean±1 SE of the percentage vasoconstriction increase over baseline. ANOVA showed significant main effects with Aβ (p<0.001), L-NAME (p=0.001), and ET-1 (p<0.001) and an interaction between ET-1 and Aβ (p<0.001). No interaction is detected between ET-1 and L-NAME (p=0.278), or among ET-1, Aβ and L-NAME (p=0.260).

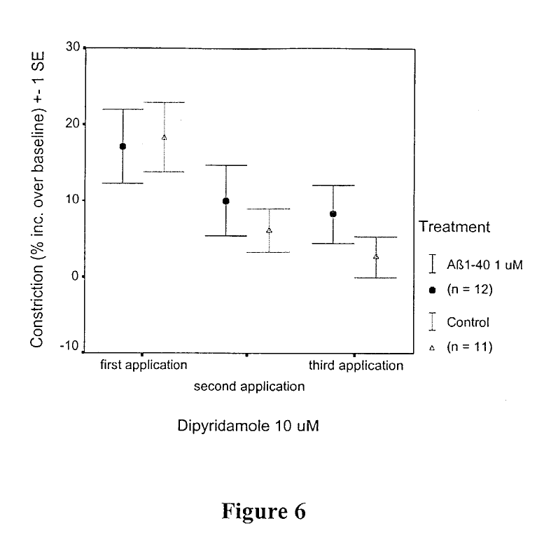

FIG. 6 is a graph demonstrating the effect of Aβ on the relaxation induced by dipyridamole. Control (N=11)and Aβ-treated vessels (N=12) are pre-constricted with 3.5 nM of PE. Following stabilization of vasotension, vessels are treated with three applications of 10 μM of dipyridamole. Data is standardized such that the maximum PE-induced constriction is fixed to 100%. ANOVA shows that dipyridamole is a significant factor (p=0.005) in relaxation. The mean relaxation induced by dipyridamole (at all applications) is not significantly different in Aβ-treated compared to control channels (p=0.258).

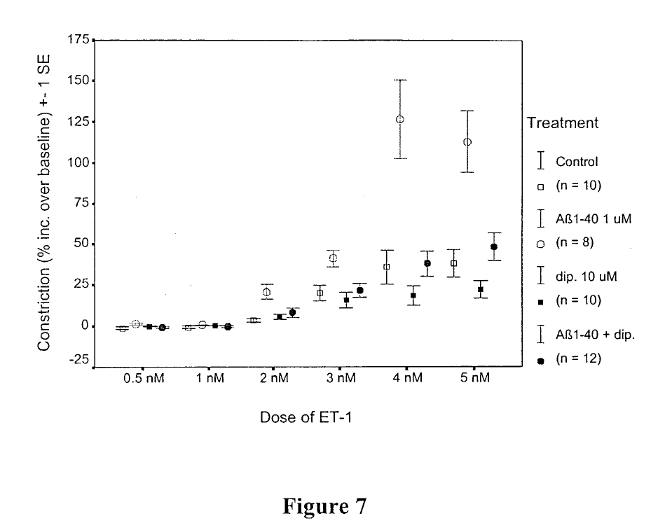

FIG. 7 is a graph showing the effect of dipyridamole on Aβ-enhancement of ET-1 induced vasoconstriction. Aortic rings are treated with a 1 μM of freshly solubilized Aβ₁₋₄₀ (N=8), 10 μM of dipyridamole (dip.) (N=10), or dip.+Aβ (N=12) for five minutes prior to the addition of a dose range of ET-1. Results are expressed as the mean±1 SE of the percentage vasoconstriction increase over baseline. There are significant main effects of ET-1 (p<0.001), Aβ (p<0.001) and dip. (p<0.001), as well as significant interactive terms between ET-1 and either Aβ (p<0.001) or dip. (p<0.001). Furthermore, there is a significant interactive term among ET-1, Aβ and dip. (p=0.001). One-way ANOVA reveals significant between treatment groups differences across all doses of ET-1 (p<0.001), and post-hoc comparison between dip. and control vessels across the 4 nM and 5 nM doses of ET-1 reveals a significant difference (p<0.05).

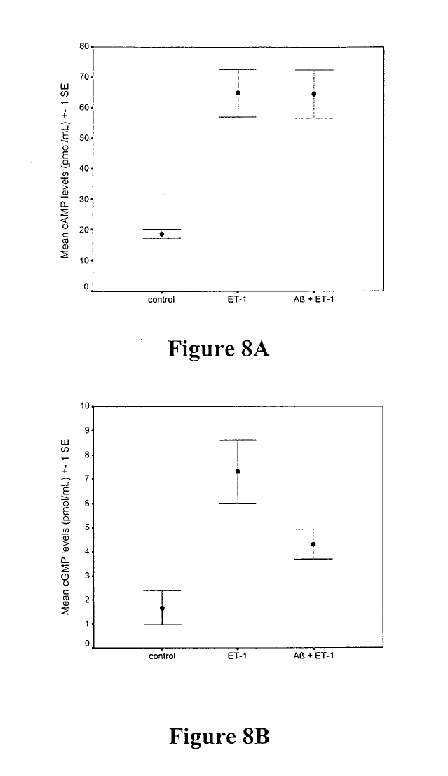

FIG. 8A is a graph showing cAMP levels in isolated rat aortae after ET-1 or Aβ+ET-1 treatment. Results are expressed as mean values (pmol/mL of cAMP)±SEs. When measuring cAMP, T-test for independent samples reveals significant differences between control and ET-1 (p<0.001), but not between ET-1 and ET-1+Aβ (p>0.05).

FIG. 8B is a graph showing cGMP levels in isolated rat aortae after ET-1 or Aβ+ET-1 treatment. Results are expressed as mean values (pmol/mL of cGMP per aortic ring)±SEs. When measuring cGMP, T-test for independent samples reveals significant differences between control and ET-1 (p<0.01), and between ET-1 and ET-1+Aβ (p<0.05)

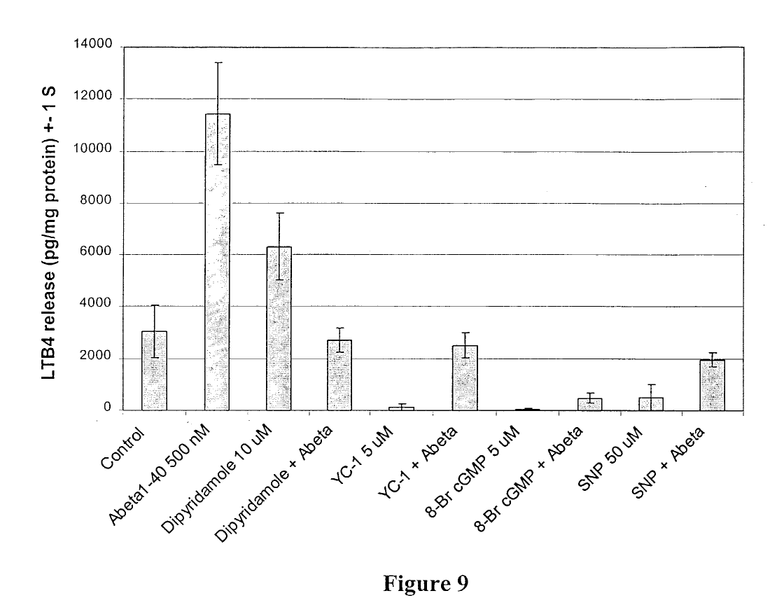

FIG. 9 is a graph demonstrating the effects of various treatment conditions on Aβ-induced microglial LTB4 release. N=6 for each condition presented. ANOVA reveals significant main effects of Aβ (p<0.01), dipyridamole (10μM, p=0.01), YC-1 (p<0.001), 8-Br cGMP (p<0.001), and SNP (p<0.001). Significant interactive terms are noted between Aβ and each drug used (p<0.01). Post-hoc testing does not reveal significant differences between control and each drug used or control and drug+Aβ (p>0.05). This shows that each drug used alone does not significantly affect constitutive LTB4 release, and administration of each drug in combination with Aβ results in total blockade of Aβ-induced microglial LTB4 release.

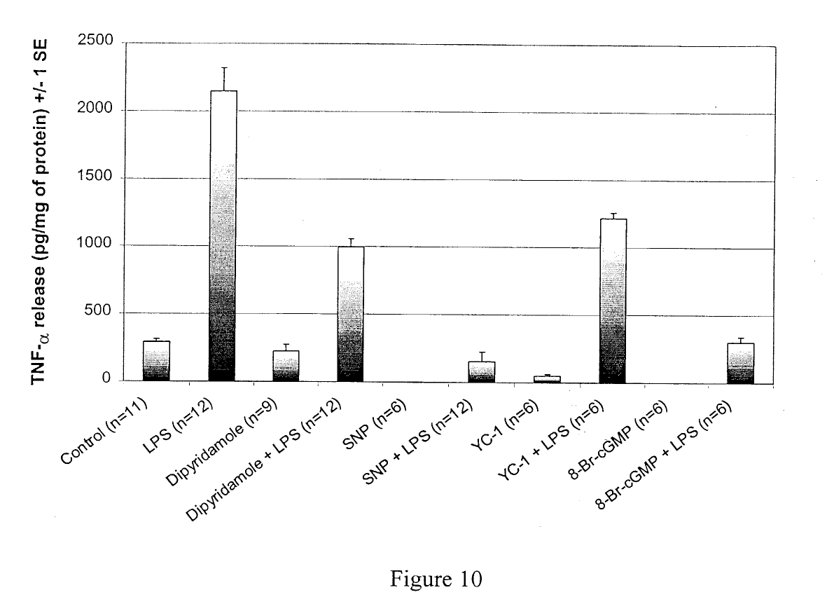

FIG. 10 is a graph showing cyclic GMP elevating agents block microglial activation induced by LPS. N9 microglia are cultured for 18 hours under the treatment conditions indicated. Results are expressed as TNF-α release in pg/mg of cellular protein±1 SE. ANOVA reveals significant main effects of LPS treatment (p<0.001), as well as for each compound used (p<0.01). There are also significant interactive terms between LPS treatment and addition of each drug (p≦0.02). One-way ANOVA reveals significant between-groups differences (p<0.001), and post-hoc comparison shows significant differences of the means between control and LPS treatment (p<0.001), between LPS treatment and LPS+drug x (p<0.001), but not between control and drug x (p>0.05).

### DETAILED DESCRIPTION OF THE INVENTION

### General

The present invention provides a method for treating AD by reducing β-amyloid vasoactivation and β-amyloid microglial mediated inflammation via inhibition of cGMP-PDE or elevation of cGMP. Treating vessels, such as the aorta, with Aβ will sensitize them to vasoconstrictors such as PE and ET-1, resulting in vasoconstriction. Further, adding a therapeutic agent that inhibits that interaction hence blocking Aβ-mediated vasoconstriction, will reduce AD pathology. Additionally, adding the therapeutic agent, then a vasoconstrictor and measuring vasoactivity indicates the therapeutic effectiveness of an agent. Therapeutic effectiveness is defined as reducing AD pathology as measured by reduced vasoconstriction, increased cGMP levels, decreased LBT4 production, or decreased TNF-αproduction.

While this invention is not to be limited by theory, Aβ may exert its vasoactive effects by decreasing the biological activity of NO, a compound that relaxes vascular smooth muscle primarily through sGC activation, rather than by modulating the amount of NO. Thus, an agent, which inhibits cGMP-PDE or elevates cGMP, will reduce Aβ vasoactivity. Vasoactivity includes vasoconstriction, decreased vasorelaxation, altered vasotonus, and other alterations in the vessel. Vasotonus and vasoconstriction are measured by a number of methods known to those of skill in the art, including measuring relaxation or constriction of a blood vessel, alterations in local blood flow, such as cerebral blood flow, and measuring the perfusion level of a tissue or bodily region. Therefore, it is demonstrated that Aβ decreases the sensitivity of sGC to NO stimulation or that Aβ opposes the effect of NO by another mechanism.

Such is the case as demonstrated in the examples below. Addition of dipyridamole, a specific cGMP-PDE inhibitor, results in reduction of vasoconstriction and enhancement of cGMP levels. By using dipyridamole, vasoconstriction is abolished as well as the opposition to relaxation normally induced by Aβ, further demonstrating that intracellular levels of cGMP are critical for mediating the vasoactive properties of Aβ, and suggesting that Aβ enhances the degradation of cGMP by stimulating cGMP-PDE.

In addition, NOS inhibition by L-NAME significantly enhances ET-1-induced vasoconstriction, however, there is no statistical interaction among ET-1, Aβ, and L-NAME, confirming that Aβ vasoactivity is not the result of NOS inhibition. Thus, these data confirm previous studies showing that the vasoactive properties of Aβ are not a result of an alteration in the production of superoxide, NO, or peroxynitrite. Therefore, these data show that Aβ does not block NO-induced cGMP synthesis, but rather, activates the degradation of cGMP via cGMPPDEs.

Indeed, by inhibiting sGC with ODQ, Aβ vasoactivity is only increased in an additive manner with ODQ treatment, suggesting that Aβ's vasoactivity is not primarily mediated by sGC. Consistent with this, stimulation of sGC with YC-1 reduces Aβ vasoactivity in an additive way, further confirming that sGC is not critically involved in Aβ-induced vasoconstriction. Although sGC or NO production are not critical mediators of Aβ vasoacticity, they may provide therapeutic benefit for Aβ as they do reduce Aβ-mediated vasoconstriction.

Inhibition of cGMP degradation by dipyridamole may block Aβ vasoactivity by effecting other signal transduction pathways. In particular, the beneficial effects of dipyridamole against Aβ vasoactivity may result from the anti-inflammatory properties displayed by cGMP-elevating agents such as phosphodiesterase inhibitors.

The pro-inflammatory response in microglia induced by Aβ is also explored within the examples provided herein.

Eicosanoids are well-known mediators of inflammation. Therefore LTB4 production, a stable eicosanoid product of the classical pro-inflammatory arachidonic acid/5-lipoxygenase cascade is measured to indicate microglial inflammation. LTB4 production in microglia is increased after Aβ treatment demonstrating that Aβ induces a pro-inflammatory response in microglia. Furthermore, dipyridamole, YC-1, cGMP and SNP block LTB4 release induced by Aβ, showing that cGMP-elevating agents display anti-inflammatory properties and block Aβ-induced inflammation in microglia. These data raise the possibility that cGMP-elevating agents may be beneficial in the treatment of other disorders that involve an inflammatory component, such as rheumatoid arthritis. In addition, dipyridamole is known to block neuronal death induced by trophic factor withdrawal suggesting that cGMP might have trophic effects on neurons. This is particularly relevant to AD as investigators have suggested that cGMP levels are also affected in AD brains.

Thus, Aβ vasoactivity and Aβ-induced microglial pro-inflammation share a similar signal transduction pathway, since drugs which block Aβ-vasoactivity also appear to be efficient inhibitors of Aβ-induced microglial inflammation. Thereby multiple methods for evaluating the therapeutic effectiveness of a therapeutic agent for the treatment of AD are provided, in particular, an agent that inhibits cGMP-PDE or increases cGMP levels.

The methods disclosed herein are easily adapted by those of skill in the art: preferred ranges of concentrations of reagent in the instant embodiments of the invention include endothelin-1, with a preferred concentration range of between 1 nM and 5 nM, and β-amyloid peptides, with a preferred concentration range of between 0.1 M and 10 M.. Many amyloidogenic peptides may be used in the present invention: in the current embodiments, various fragments of β-amyloid are used, including, but not limited to, Aβ₁₋₄₂, Aβ₁₋₄₁, Aβ₁₋₄₀, Aβ₁₋₄₃, Aβ₁₋₂₈, Aβ₂₅₋₃₅, and derivatives of these, where derivitization can include amino acid substitutions, glycosylation, and the like.

### EXAMPLE 1

5.21 Materials and Methods

Aβ₁₋₄₀ is supplied by QCB. ODQ, endothelin-1, phenylephrine, dipyridamole and 8-Br cGMP are obtained from Sigma. Sodium nitroprusside, N-ω-nitro-L-arginine methyl ester (L-NAME), YC-1 and competitive binding enzyme irniunoassay (EIA) cAMP and cGMP kits are purchased from Alexis Biochemicals. LTB4 competitive binding EIA kits are obtained from R&D.

Vessel Experiments

Vasoactivity is measured in rat aortic rings using the system previously described, for example by Crawford, F., et al., The vasoactivity of Aβ peptides, Ann. N. Y. Acad. Sci. 826:3546, (1997); Crawford, F., et al., Characteristics of the in vitro vasoactivity of beta-amyloid peptides, Exp. Neurol. 150:159-168 (1998); Paris, D., et al., Isoform-specific vasoconstriction induced by Apolipoprotein E and modulation of this effect by Alzheimer's β-amyloid peptide. Neurosci. Lett. 256:73-76, (1998); Paris, D., et al., Role of peroxynitrite in the vasoactive and cytotoxic properties of Alzheimer's β-amyloid₁₋₄₀ peptide, Exp. Neurol. 152:116-122 (1998); and Thomas, T. N., et al., Vasoactive effects and free radical generation by β-amyloid peptides. U.S. Pat. No. 6,011,019. Jan. 4, 2000. Normal male Sprague-Dawley rats (7-8 months old) are sacrificed, and freshly dissected rat aortae are segmented into 3 mm rings and suspended in Kreb's buffer on hooks. These hooks are connected to an isometric transducer linked to a MacLab system. Aortic rings are equilibrated in the tissue bath system for two hours with the Kreb's buffer changed every 30 min.

For the vasoconstriction assay, the first group of aortic rings are pre-treated with 10 μM ODQ 2 minutes prior to the addition of 1 μM of Aβ₁₋₄₀. After 5 minutes of incubation with Aβ, the vessels are then subjected to a dose range of ET-1 (from 1 nM to 5 nM). The second set of vessels are treated with Aβ prior to the addition of ET-1. A third set received ODQ treatment followed by ET-1, and the fourth group (control) only received ET-1 treatment. A similar protocol is applied with dipyridamole (10 μM), and YC-1 (5 μM). In all cases the percentage contraction as compared to baseline is determined for each dose of ET-1 used. The means and standard errors (SEs) of all such values are calculated.

For the vasorelaxation assay, constricted aortic rings are pre-treated for 5 minutes with 1 μM of Aβ with a single dose of phenylephrine (3.5×10⁻⁹ M) along with untreated controls. After waiting for stabilization of vasotension, aortic segments are subjected to a range of SNP doses. Aortic rings pre-constricted with a single dose of phenylephrine (3.5×10⁻⁹ M) are treated with 10 μM ODQ 5 minutes prior to the addition of several doses of SNP. The effect of 10 μM of dipyridamole is also investigated on vessels pre-constricted with 3.5×10⁻⁹ M of phenylephrine. Data are standardized such that the maximum PE-induced constriction is fixed at 100%.

5.22 Results

Effect of Sodium Nitroprusside on Aβ Pre-treated Rat Aortae

In order to obtain a stable, long-lasting vasoconstriction event, vessels are treated with phenylephrine (PE). In aortic rings treated with Aβ and constricted with PE, we show that the relaxation induced by SNP, an NO-donor, is reduced in comparison to the relaxation induced by the same amount of SNP in control rings (FIG. 1). These data suggest either that Aβ decreases the sensitivity of sGC to NO stimulation, or that Aβ opposes the effects of NO via another mechanism.

Effect of Inhibition and Stimulation of the NO/sGC/cGMP Pathway on Aβ vasoactivity

The role of sGC sensitivity to NO in the Aβ vasoactivity paradigm is then investigated. To assess the effects of sGC inhibition on Aβ vasoactivity, ODQ, a highly selective inhibitor of sGC is used in the rat aorta assay. The effect of SNP on rat aortae treated with ODQ shows that ODQ blocks the relaxation induced by this NO donor (FIG. 2). These data demonstrate that, although NO stimulates relaxation through cGMP-dependent and -independent pathways, the predominant pathway in rat aortae is mediated via the production of cGMP.

Since the ET-1 signal transduction pathway involves activation of the NO/cGMP pathway, the effects of ODQ and Aβ on the vasoconstriction induced by ET-1 are examined. Neither ODQ or Aβ alone are able to potentiate vasoconstriction induced by ET-1 in an ET-1 dose-dependent (statistically interactive) manner (FIG. 3) in this assay. However, co-treatment of the aortic rings with ET-1, Aβ and ODQ gives constriction which is only additive, suggesting that sGC is not modulated by Aβ (FIG. 3).

To confirm this result, rat aortic rings are treated with YC-1, a NO-independent activator of sGC. This results in a statistically interactive reduction of ET-1 vasoconstriction; yet, the observed reduction of Aβ vasoactivity is merely additive, showing that Aβ vasoactivity is not mediated via inhibition of sGC (FIG. 4).

Next, the involvement of NOS in Aβ vasoactivity is examined by inhibiting NOS with L-NAME. NOS inhibition does not significantly increase ET-1-induced vasoconstriction and, as no statistical interaction is observed among ET-1, Aβ and L-NAME (FIG. 5), this suggests that Aβ vasoactivity is not due to an alteration of NOS activity. This data demonstrates that NO is not an important mediator of Aβ vasoactivity.

As Aβ does not induce vasoactivity through inhibition of sGC or NOS, however, Aβ opposes the relaxation induced by NO (FIG. 1), Aβ may decrease the level of intracellular cGMP by enhancing cGMP degradation primarily controlled by cGMP-PDEs. Therefore, involvement of type V cGMP-PDE in the rat aorta assay is examined.

Effect of type V cGMP-PDE Inhibition on the Vasoactivity triggered by Aβ

Type V cGMP-PDE is inhibited by pre-treating rat aortae with the selective inhibitor dipyridamole, see for example, Farinelli, S. E., et al., Nitric oxide delays the death of trophic factor-deprived PC12 cells and sympathetic neurons by a cGMP-mediated mechanism. J. Neurosci. 16:2325-2334, (1996); and Vroom, M. B. et al., Effect of phosphodiesterase inhibitors on human arteries in vitro. Brit. J. Anaesth. 76:122-129 (1996). On quiescent rings, 10 μM of dipyridamole have no significant relaxant effect (data not shown), whereas on aortic rings constricted with phenylephrine, dipyridamole induced potent relaxation (FIG. 6). Following constriction with PE, dipyridamole-induce relaxation between Aβ-treated and control channels is not significantly different, showing that dipyridamole blocks the opposition to relaxation normally induced by Aβ (see FIG. 6).

Investigation of the effects of dipyridamole on Aβ-enhancement of ET-1-induced vasoconstriction shows that Aβ enhances ET-1-induced vasoconstriction in an ET-1- dose-dependent manner. Additionally, dipyridamole opposes ET-1-induced vasoconstriction in an ET-1 dose-dependent manner. Indeed, dipyridamole blocks Aβ-induced vasoconstriction in a statistically interactive manner (by ANOVA, among ET-1, Aβ and dipyridamole) suggesting that Aβ vasoactivity is mediated through an activation of cGMP-PDE (FIG. 7).

5.23 Statistical Analysis

Analysis of variance (ANOVA) and Scheffe's post-hoc test is used for multiple comparison of the means where appropriate. T-test for independent samples is used for single mean comparisons. Alpha levels for each analysis are set at 0.05. All analyses are performed using SPSS for windows release 7.5.1. As previously described (Paris, D., et al., Soluble beta-amyloid peptides mediate vasoactivity via activation of a pro-inflammatory pathway, Neurobiol Aging March-April 2000, 21 (2); 183-97), a significant interactive term by ANOVA is taken as evidence that both drug x and Aβ are modulating a common signal transduction pathway.

### EXAMPLE 2

5.31 Materials and Methods

Quantification of cGMP and cAMP Levels in Rat Aortae

Aortic rings are equilibrated in Kreb's buffer for 2 hours and then constricted with a serial dose range of ET-1 (from 1 nM to 5 nM) in the presence or absence of 1 μM of Aβ. Immediately following maximum vasoconstriction (or, in the case of control rings, after 2 hours of equilibration in Kreb's buffer), aortae are frozen in liquid nitrogen. Vessels are then homogenized in 10% trichloracetic acid (TCA) diluted in PBS (at 4° C.) and centrifuged to pellet TCA precipitated proteins. Supernatants are collected and TCA is then removed from samples by three extractions with diethyl ether saturated in deionized water. Residual ether is removed from the sample by heating samples to 70° C. for 10 minutes. Both cGMP and cAMP levels are quantified using competitive binding EIAs (Alexis Biochemicals, San Diego, Calif.) according to the manufacturer's instruction.

5.32 Results

Measurement of cGMP and cAMP Levels in Rat Aortic Rings

To determine whether Aβ-mediated vasoactivity relies on an enhanced degradation of cGMP, levels of cGMP are quantified in rat aortic rings treated with ET-1 alone, ET-1+Aβ, or control (untreated vessels). In order to determine whether the Aβ effect is specific to cGMP, both cGMP and cAMP levels are measured in the same extracts. ET-1 treatment results in an increase in cAMP and cGMP levels. Further Aβ is not able to modulate cAMP levels in conjunction with ET-1 (FIG. 8a). Yet, in Aβ and ET-1 co-treated vessels, cGMP levels are significantly lower compared to ET-1 treatment alone further demonstrating that cGMP degradation is enhanced by Aβ and ET-1 co-treatment in rat aorta (FIG. 8b).

5.33 Statistical Analysis

Analysis of variance (ANOVA) and Scheffe's post-hoc test is used for multiple comparisons of the means where appropriate. T-test for independent samples is used for single mean comparisons. Alpha levels for each analysis are set at 0.05. All analyses are performed using SPSS for windows release 7.5.1. As previously described (Paris, D., et al., Soluble beta-amyloid peptides mediate vasoactivity via activation of a pro-inflammatory pathway, Neurobiol Aging March-April 2000, 21 (2); 183-97), a significant interactive term by ANOVA is taken as evidence that both drug x and Aβ are modulating a common signal transduction pathway.

### EXAMPLE 3

5.41 Materials and Methods

Microglial Cell Culture

The murine microglial cell line (N9) is kindly provided by Dr. Paola Ricciardi-Castagnoli (Cellular Pharmacology Center, Milan, Italy) and cells are grown in RPMI 1640 medium supplemented with 5% fetal calf serum, 2 mM glutamine, 100 U/mL penicillin, 0.1 μg/mL streptomycin and 0.05 mM 2-mercaptoethanol. Microglial cells are seeded at 50,000 cells/well in 6-well plates (Falcon, France) and treated with Aβ₁₋₄₀ (500 nM), dipyridamole (10 μM) or untreated (control) and incubated for 18 hours. Cell supernatants are then collected and immediately frozen at −80° C.

Quantification of LTB4 Release from Microglial Cells

Cell supernatants (50 μL) are used in the LTB4 assay, and each sample is assayed in duplicate. Manipulations are performed in accordance with the manufacturer's instruction. A spectramax 250 spectrophotometer (Molecular Devices, San Diego, USA) is used to measure absorbance at 405 nm and a standard curve is plotted using a 4-parameter model. Cell extracts for protein determination are obtained by lysing microglial cells in 110 μL of ice cold lysis buffer (20 nM Tris pH 7.5, 150 mM NaCl, 1 mM EDTA, 1 mM EGTA, 1% Triton X-100, 2.5 mM sodium pyrophosphate, 1 mM β-Glycerophosphate, 1 mM Na₃VO₄, 1 μg/ml leupeptin and 1 mM phenylmethylsulfonyl fluoride). Protein concentrations are determined for each sample using the Biorad reagent according to the manufacturer's instruction. LTB4 data are thus expressed as pg LTB4 /mg cellular protein.

5.42 Results

Effect of cGMP-elevating Compounds on Aβ-induced Microglial Inflammation

Increasing evidence has implicated inflammation as a contributor to the pathology of AD. Non-steroidal anti-inflammatory drugs (NSAIDs) have been shown to be prophylactic against the cognitive decline associated with aging and AD. Although the precise role of inflammation in AD pathogenesis is not known, the association of immune system proteins and reactive gliosis with senile plaques suggests a possible involvement of Aβ in the induction of this inflammatory process. Thus the pro-inflammatory effects of Aβ on microglia are examined by measuring LTB4 production. LTB4 is selected because it is a stable eicosanoid product of the classical pro-inflammatory arachidonic acid/5-lipoxygenase cascade, as are other derivatives of arachidonic acid. Freshly solubilized Aβ₁₋₄₀ (500 nM) induces a pro-inflammatory response in microglia as evidenced by significant LTB4 release in Aβ-treated versus untreated cells (FIG. 9).

As Aβ may mediate pro-inflammation in micro,glia and vasoactivity through a similar signal transduction pathway, the effects of dipyridamole and other cGMP-elevating compounds on Aβ induced microglial inflammation are examined. Dipyridamole completely prevents the increased LTB4 production induced by Aβ (FIG. 9). Moreover, 8-bromo-cGMP, a membrane permeable cGMP analogue, YC-1 and SNP also completely block Aβ-induced microglial LTB4 release (FIG. 9), suggesting that cGMP-elevating agents, in particular NO, display anti-inflammatory properties. This data shows that dipyridamole blocks both Aβ-induced vasoconstriction and microglial LTB4 release via a cGMP-dependent mechanism, showing that Aβ mediates its bioactivity through a common signal transduction pathway in different cell types.

5.43 Statistical Analysis

Analysis of variance (ANOVA) and Scheffe's post-hoc test is used for multiple comparison of the means where appropriate. T-test for independent samples is used for single mean comparisons. Alpha levels for each analysis are set at 0.05. All analyses are performed using SPSS for windows release 7.5.1. As previously described (Paris, D., et al., Soluble beta-amyloid peptides mediate vasoactivity via activation of a pro-inflammatory pathway, Neurobiol Aging March-April 2000, 21 (2); 183-97), a significant interactive term by ANOVA is taken as evidence that both drug x and Aβ are modulating a common signal transduction pathway.

### EXAMPLE 4

5.51 Materials and Methods

The N9 murine microglial cell line is kindly provided by Dr. Paola Ricciardi-Castagnoli (Cellular Pharmacology Center, Milan, Italy), and microglia are grown in RPMI 1640 medium supplemented with 5% fetal calf serum, 2 mM glutamine, 100 U/mL penicillin, 0.1 μg/mL streptomycin, and 0.05 mM 2-mercaptoethanol. Microglial cells are seeded at 5×10⁴ cells/well in 6-well tissue-culture plates (Falcon, France), and are subjected to LPS treatment (2.5 ng/mL) or untreated (control), in the presence or absence of various pharmacological agents for 18 h. Just prior to seeding in 6-well tissue-culture plates, microglia are assayed for TNF-α production to assure that these cells had not become spontaneously activated (microglia are utilized only when TNF-α production is <400 pg/mg total protein), an effect previously observed in “aged” microglial cell cultures [13]. Cell supernatants are collected and immediately frozen at −80° C. TNF-α levels are determined using the mouse TNF-α DUOSET™ ELISA kit (Genzyme, Cambridge, Mass.) in accordance with the manufacturer's instruction. Cell extracts for protein quantification are obtained by lysing viable, adherent microglial cells in 110 μL of ice-cold lysis buffer (containing 20 mM Tris, pH 7.5, 150 mM NaCl, 1 mM EDTA, 1 mM EGTA, 1% Triton X-100, 2.5 mM sodium pyrophosphate, 1 mM β-glycerophosphate, 1 mM Na₃VO₄, 1 μg/mL leupeptin, and 1 mM phenylmethylsulfonyl fluoride). Cellular protein concentrations are determined for each sample using the Bio-Rad reagent (Bio-Rad Laboratories, Hercules, Calif.) according to the manufacturer's instruction. YC-1 is purchased from CALBIOCHEM (San Diego, Calif.). LPS, from E. coli 026:B6, the cGMP analogue 8-Br cGMP, and the cGMP-elevating agents dipyridamole and SNP are purchased from Sigma.

In order to investigate the role of the nitric oxide/cGMP (NO/cGMP) signaling pathway on LPS-induced microglial activation, N9 microglia are co-incubated with LPS and various compounds that act by increasing intracellular cGMP. As shown in FIG. 10, the cGMP-elevating agents tested, including dipyridamole, SNP, and YC-1, markedly reduce microglial activation induced by LPS. Moreover, 8-Br cGMP at 5 μM appears to completely inhibit LPS-induced microglial TNF-α release (at 1 μM 8-Br cGMP is able to partially inhibit LPS-induced microglial TNF-α release, data not shown). Each of these effects is statistically interactive by ANOVA, suggesting that stimulation of the NO/cGMP pathway negatively regulates LPS-induced microglial TNF-α production.

Whether the effects of cGMP-elevating agents may impede LPS-induced microglial TNF-α release are then determined. Thus, a cell-freely permeable cGMP analogue, 8-bromo cGMP (8-Br cGMP, 5 μM), and cGMP-elevating agents including the nitric oxide donor, sodium nitroprusside (SNP, 10 μM), a nitric oxide-independent activator of soluble guanylyl cyclase, YC-1 (10 μM), and an inhibitor of the cGMP-specific phosphodiesterase type V (dipyridamole, 10 μM) are used in the microglial assay.

This data shows that NO mediates its effect via activation of soluble guanylyl cyclase, resulting in strengthening of intracellular cGMP signaling demonstrating an anti-inflammatory role of cGMP, and showing that cGMP-elevating agents oppose microglial activation as well as pro-inflammation in microglia cells.

All publications, patents, and patent documents referred to herein are hereby incorporated in their respective entireties by reference.

The invention has been described with reference to the foregoing specific and preferred embodiments and methods. However, it should be understood that many variations may be made while remaining within the spirit and scope of the invention. Therefore, the foregoing examples are not limiting, and the scope of the invention is intended to be limited only by the following claims.

What is claimed is:

1. A method of screening for a compound for use in the treatment of a neurodegenerative disease, said method comprising:
contacting a blood vessel with a β-amyloid peptide, a vasoconstrictor, and said compound; and
measuring vasoactivity of said blood vessel.
